(19) United States
(12) Patent Application Publication
(10) Pub. No.: US 2008/0293038 A1
(43) Pub. Date: Nov. 27, 2008

(54) Method for Determining Resistance of Hiv to Nucleoside Reverse Transcriptase Inhibitor Treatment

(76) Inventors: Neil T. Parkin, Belmont, CA (US); Colombe Chappey, San Francisco, CA (US); Jeannette Whitcomb, San Mateo, CA (US)

Correspondence Address: JONES DAY, 222 EAST 41ST ST, NEW YORK, NY, 10017, US

(21) Appl. No.: 11/915,307

(22) PCT Filed: May 25, 2006
(86) PCT No.: PCT/US06/20364

§ 371 Date: Jul. 11, 2008

Related U.S. Application Data
(60) Provisional application No. 60/685,337, filed on May 27, 2005.

(51) Int. Cl. C12Q 1/70

(52) U.S. Cl. 435/5

(57) ABSTRACT

The present invention provides methods and devices for predicting whether an HIV-1 is resistant to an antiviral drug based on the HIV-1's genotype. In one aspect, the invention provides methods comprising determining whether a mutation or combination of mutations associated with NRTI resistance are present, as disclosed herein, thereby assessing the effectiveness of FTC therapy in the HIV-infected subject. Computer implemented methods comprising determining HIV-1 resistance are provided.

### 1. FIELD OF THE INVENTION

This invention relates to methods and devices for determining the susceptibility of a pathogenic virus to an anti-viral compound. In particular, this invention relates to methods and devices useful for the identification of HIV resistance to nucleoside reverse transcriptase inhibitor (“NRTI”), e.g., emtricitabine (“FTC”), therapy in a subject infected with HIV using genotypic information of the HIV.

### 2. BACKGROUND OF THE INVENTION

More than 60 million people have been infected with the human immunodeficiency virus (“HIV”), the causative agent of acquired immune deficiency syndrome (“AIDS”), since the early 1980s. See Lucas, 2002, Lepr Rev. 73(1):64-71. HIV/AIDS is now the leading cause of death in sub-Saharan Africa, and is the fourth biggest killer worldwide. At the end of 2001, an estimated 40 million people were living with HIV globally. See Norris, 2002, Radiol Technol. 73(4):339-363.

The goal of antiretroviral therapy drug treatment is to delay disease progression and prolong survival by achieving sustained suppression of viral replication. Current anti-HIV drugs target different stages of the HIV life cycle and a variety of enzymes essential for HIV's replication and/or survival. For example, certain drugs approved for AIDS therapy inhibit HIV replication by interfering with the enzymatic activities of either protease (“PR”) or reverse transcriptase (“RT”). Amongst the approved drugs are NRTIs such as AZT, ddI, ddC, d4T, 3TC, abacavir, nucleotide reverse transcriptase inhibitors such as tenofovir, non-nucleoside reverse transcriptase inhibitors (“NNRTIs”) such as nevirapine, efavirenz, delavirdine and protease inhibitors (“PIs”) such as saquinavir, ritonavir, indinavir, nelfinavir, amprenavir and lopinavir.

One consequence of the action of an anti-viral drug is that it can exert sufficient selective pressure on virus replication to select for drug-resistant mutants. Herrmann et al., 1977, Ann NY Acad Sci 284:632-637. With increasing drug exposure, the selective pressure on the replicating virus population increases to promote the more rapid emergence of drug resistant mutants.

With the inevitable emergence of drug resistance, strategies must be designed to optimize treatment in the face of resistant virus populations. Ascertaining the contribution of drug resistance to drug failure is difficult because patients that are likely to develop drug resistance are also likely to have other factors that predispose them to a poor prognosis. Richman, 1994, AIDS Res Hum Retroviruses 10:901-905. In addition, each patient typically harbors a diverse mixture of mutant strains of the virus with different mutant strains having different susceptibilities to anti-viral drugs.

Antiviral drug susceptibility assays for clinical HIV isolates are required to monitor the development of drug resistance during therapy. Ideally, assays that determine the drug susceptibility of HIV isolates should be rapid, reproducible, non-hazardous, applicable to all samples, and cost-effective. Two general approaches are now used for measuring resistance to anti-viral drugs. The first approach, called phenotypic testing, measures the susceptibility of virus taken from an infected person's virus to particular anti-viral drugs in an in vitro assay system. See, e.g., Kellam & Larder, 1994, Antimicrobial Agents and Chemo. 38:23-30; Petropoulos et al., 2000, Antimicrob. Agents Chemother. 44:920-928; Hertogs et al., 1998, Antimicrob Agents Chemother 42(2):269-76. The second approach, genotypic testing, involves identifying the presence of mutations in the HIV nucleic acid that confer resistance to certain antiviral drugs in a patient infected with that virus.

Genotypic testing, in some aspects, promises certain advantages over phenotypic testing since the facilities necessary for genotypic testing are generally cheaper and less complex than those for phenotypic testing, and genotyping is typically less labor intensive to perform and results can be had in less time. However, in order to deduce the viral sensitivity from a given genotype, the effect on drug resistance of particular resistance mutations need to be known. An additional complication of gentoypic assays is that the manual interpretation of such assays is difficult because a large number of drug resistance mutations interact in complex patterns.

Therefore, need exists not only for assessing the pertinent set of mutations relevant to a given antiviral drug therapy, but methods and devices that apply rules assigning a level of resistance to a drug or drug combination on the basis of a pattern of mutations. However, no robust genotypic correlates of reduced susceptibility to 3TC or FTC therapy have been defined. As such, no robust genotypic assay with defined algorithms is presently available for assessing the efficacy of 3TC or FTC treatment in an HIV-1-infected patient. These and other needs are satisfied by the present invention.

### 3. SUMMARY OF THE INVENTION

In certain aspects, the present invention provides a method of determining whether an HIV-1 is likely to be resistant to a nucleoside reverse transcriptase inhibitor (“NRTI”), comprising detecting in a gene encoding reverse transcriptase of the HIV-1 one or more primary mutations or at least four secondary mutations, wherein the primary mutations are selected from the group consisting of mutations in codons 65, 151, and 184 and an insertion at codon 69, and the secondary mutations are selected from the group consisting of mutations at codons 41, 44, 67, 70, 118, 210, 215, and 219. In certain embodiments, the primary mutations are selected from the group consisting of K65R, Q151M, M184I, M184V, M184T, and any insertion of one or more amino acids at position 69. In certain embodiments, the secondary mutations are selected from the group consisting of M41L, E44A, E44D, D67N, K70R, V118I, L210W, T215F, T215Y, K219E, K19H, K219N, K219Q and K219R.

In certain embodiments, the NRTI is FTC.

In certain embodiments, the HIV-1 is an HIV-1 isolated from a patient sample. In certain embodiments, the HIV-1 is isolated from the patient sample without passage through cell culture.

In certain embodiments, the HIV-1 determined to have a likelihood for reduced NRTI susceptibility exhibits a 3.5-fold change in a PHENOSENSE™ phentotypic HIV-1 assay compared to a reference HIV-1.

In certain embodiments, the reference HIV-1 is the NL4-3 strain of HIV-1.

In certain embodiments, the NRTI is FTC, the primary mutation is selected from the group consisting of K65R, Q151M, M184I, M184V, M184T, and any insertion of one or more amino acids at position 69, and the secondary mutations are selected from the group consisting of M41L, E44A, E44D, D67N, K70R, V118I, L210W, T215F, T215Y, K219E, K219H, K219N, K219Q and K219R.

In another aspect, the present invention provides a method for assessing the effectiveness of FTC therapy in a HIV-1-infected subject comprising detecting in a gene encoding reverse transcriptase of the HIV-1 one or more primary mutations or at least four secondary mutations, wherein the primary mutations are selected from the group consisting of mutations in codons 65, 151, and 184 and an insertion at codon 69, and the secondary mutations are selected from the group consisting of mutations at codons 41, 44, 67, 70, 118, 210, 215, and 219, thereby assessing the effectiveness of FTC therapy in the subject. In certain embodiments, the primary mutations are selected from the group consisting of K65R, Q151M, M184I, M184V, M184T, and any insertion of one or more amino acids at position 69. In certain embodiments, the secondary mutations are selected from the group consisting of M41L, E44A, E44D, D67N, K70R, V118I, L210W, T215F, T215Y, K219E, K219H, K219N, K219Q and K219R.

In certain embodiments, the decrease in susceptibility to FTC therapy is at least 3.5-fold.

In one aspect, the present invention provides a computer implemented method of determining that an HIV-1 is likely to be resistant to FTC, comprising inputting to a computer-readable medium a genotype of HIV-1 reverse transcriptase from the HIV-1 and comparing the genotype of the HIV-1 reverse transcriptase to a database in a computer-readable medium that comprises a correlation between the presence of a mutation at 65, 151, or 184 or an insertion at codon 69 and resistance to FTC, wherein if the comparison identifies that a mutation correlated with FTC resistance is present, the HIV-1 is determined to resistant to FTC. In certain embodiments, the mutation is K65R, Q151M, M184I, M184V, M184T, or any insertion of one or more amino acids at position 69.

In certain embodiments, the methods further comprise comparing the genotype of the HIV-1 reverse transcriptase to a database in the computer system that comprises a correlation between the presence of a mutation in at least four of codons 41, 44, 67, 70, 118, 210, 215, and 219 and resistance to FTC, wherein if the comparison identifies that a mutation correlated with FTC resistance is present, the HIV-1 is determined to resistant to FTC. In certain embodiments, the mutation is M41L, E44A, E44D, D67N, K70R, V118I, L210W, T215F, T215Y, K219E, K219H, K219N, K219Q or K219R.

In certain embodiments, the computer implemented method further comprises displaying whether or not the HIV-1 is determined to be resistant to FTC. For example, the result may be displayed on a tangible medium such as paper or other form of printout or on a computer screen, or other tangible media without limitation.

In another aspect, the invention provides an article of manufacture that comprises computer-readable instructions for performing a computer implemented method of the invention. For example, the article of manufacture can be a floppy disk, CD, DVD, magnetic tape, and so forth, without limitation.

In another aspect, the present invention provides a computer system that is configured to perform a computer implemented method of the invention.

In another aspect, the present invention provides a computer-readable medium comprising a computer program that determines whether an HIV-1 is resistant to FTC, said program comprising a computer code that receives input corresponding to the genotype of the HIV-1 nucleic acid encoding HIV-1 reverse transcriptase-obtained from the subject; a computer code that performs a first comparison to determine if codon 65, 69, 151, or 184 of the nucleic acid encoding HIV-1 reverse transcriptase is K65R, Q151M, M184I, M184V, M184T, or any insertion of one or more amino acids at position 69; a computer code that performs a second comparison to determine if codon 41, 44, 67, 70, 118, 210, 215, or 219 of the nucleic acid encoding HIV-1 reverse transcriptase is M41L, E44A, E44D, D67N, K70R, V118I, L210W, T215F, T215Y, K219E, K219H, K219N, K219Q or K219R; a computer code that determines whether at least one match is made in the first comparison or at least four matches are made in the second comparison, wherein the HIV-1 is determined to be resistant to FTC if at least one match is made in the first comparison or at least four matches are made in the second comparison; and a computer code that conveys a result representing whether or not the HIV-1 is determined to be resistant to FTC to an output device. By first and second comparisons, it should be noted that these labels serve only to distinguish the first comparison from the second comparison, and in no way indicate that order in which the comparisons are performed. Therefore, the first comparison can be performed before the second comparison, the second comparison can be performed before the first comparison, or the first and second comparisons can be performed concurrently.

In certain embodiments, the output device is a printer. In certain embodiments, the output device is a computer display, e.g., a flat panel display or a CRT monitor.

In another aspect, the invention provides a tangible medium comprising the result conveyed to the output device by the computer program product described above. In certain embodiments, the tangible medium is a printout. In other embodiments, the tangible medium is a CD or DVD.

In another aspect, the invention provides a computer-readable medium medium comprising data indicating whether an HIV-1 is resistant to FTC and computer-readable instructions for performing a method of the invention as described herein. In certain embodiments, the tangible medium is a floppy disk, CD, DVD, magnetic tape, fixed disk drive, iPod™, and the like.

### 4. DETAILED DESCRIPTION OF THE INVENTION

The present invention provides methods and devices for identifying HIV-1 populations that are resistant to an NRTI using a genotype interpretation algorithm. In these methods, the genotype of a HIV-1 is compared to a set of primary and secondary mutations that correlate with resistance to an NRTI, as described below, and depending on the number and type (i.e., primary or secondary) of matches recited by the algorithm, the HIV-1 can be categorized as being resistant or susceptible to an NRTI, e.g., FTC. Evidence presented herein indicate that the application of the newly created algorithm to the set of primary and secondary reverse transcriptase mutations correctly determines whether a HIV-1 exhibits an FC for FTC of at least 3.5 approximately 95 out of 100 times.

### 4.1 Abbreviations

“HIV” is an abbreviation for human immunodeficiency virus. “PR” is an abbreviation for protease. “PI” is an abbreviation for protease inhibitor. “PCR” is an abbreviation for polymerase chain reaction. “3TC” is an abbreviation for the NRTI lamivudine. “FTC” is an abbreviation for the emtricitabine. “FC” is an abbreviation for fold change. “GR,” “GS,” “PR” and “PS” are abbreviations for genotypically resistant, genotypically susceptible, phenotypically resistant and phenotypically susceptible, respectively.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when polypeptide sequences are presented as a series of one-letter and/or three-letter abbreviations, the sequences are presented in the N-terminus to C-terminus direction, in accordance with common practice.

Substituted or mutant amino acids in HIV-1 reverse transcriptase positions are represented herein in an abbreviated fashion such as “M184I/L/V,” where “M” is single-letter representation of the non-mutant reference amino acid methionine at position “184” of HIV-1 reverse transcriptase, and “I,” “L” and “V” represent single-letter representations of possible mutant amino acids that may be substituted for M at position 36 in the reverse transcriptase.

### 4.2 Definitions

As used herein, “genotypic data” are data about the genotype of, for example, a virus. Examples of genotypic data include, but are not limited to, the nucleotide or amino acid sequence of a virus, a part of a virus, a viral gene, a part of a viral gene, or the identity of one or more nucleotides or amino acid residues in a viral nucleic acid or protein.

Unless otherwise specified, “primary mutations” are single amino acid changes to HIV-1 RT at positions 65, 151, and 184 and any insertion at position 69, and “secondary mutations” are single amino acid changes to HIV-1 RT at positions 41, 44, 67, 70, 118, 210, 215, and 219. In certain embodiments, primary mutations are K65R, Q151M, M184I, M184V, M184T, or any insertion of one or more amino acids at position 69 and secondary mutations are M41L, E44A, E44D, D67N, K70R, V118I, L210W, T215F, T215Y, K219E, K219H, K19N, K219Q or K219R.

A “reference HIV” as used herein, is a HIV known to those of skill in the art to be a well-characterized drug-sensitive virus. For example, a reference HIV is NL4-3 (GenBank accession no. AF324493, incorporated by reference in its entirety).

“Susceptibility” refers to a virus' response to a particular drug. A virus that is less susceptible or has decreased susceptibility to a drug is less sensitive or more resistant to the drug. A virus that has increased or enhanced or greater susceptibility to a drug has an increased sensitivity or decreased resistance to the drug.

Phenotypic drug susceptibility is measured as the concentration of drug required to inhibit virus replication by 50% (IC₅₀). As used herein, a “fold change” or “FC” is the ratio of a viral variant IC₅₀ divided by the IC₅₀ of a reference HIV. An FC of 1.0 indicates that the viral variant exhibits the same degree of drug susceptibility as the reference virus.

For drugs where sufficient clinical outcome data have been gathered, it is possible to define a clinical threshold or cutoff value. A clinical threshold or cutoff value defines the point above which the utility of a given drug begins to decline based on virologic response data from clinical trials. It represents a point of increasing resistance and decreasing sensitivity of the HIV to a particular drug. The cutoff value is different for different anti-viral agents. Clinical cutoff values are determined in clinical trials by evaluating resistance and outcome data. Drug susceptibility is measured at treatment initiation. Treatment response, such as change in viral load, is monitored at predetermined time points through the course of the treatment. The drug susceptibility is correlated with treatment response and the clinical cutoff value is determined by resistance levels associated with treatment failure (statistical analysis of overall trial results).

The clinical cutoff has been identified as a 3.5-fold change (“FC”) for the PHENOSENSE™ phenotypic HIV assay for FTC. With respect to HIV populations identified or determined to be “less susceptible” or to be “resistant,” for example, less susceptible, or resistant, to FTC therapy in a subject, as used herein, such HIV populations generally meet or exceed a 3.5-fold change.

The terms “peptide,” “polypeptide” and “protein” are used interchangeably throughout.

The terms “polynucleotide,” “oligonucleotide” and “nucleic acid” are used interchangeably throughout.

The term “concordance” as used herein, means that a genotype from an HIV sample categorized as GR or GS according to an algorithm matches the phenotype (PR or PS) of that HIV sample.

The term “discordance” as used herein, means that a genotype from an HIV sample categorized as GR or GS according to an algorithm does not match the phenotype of the that HIV sample. Discordance samples include both false negatives (GS-PR) and false positive (GR-PS) identifications.

The methods and devices of the present invention arise, in part, out of the creation of an algorithm that predicts HIV resistance to FTC based on a HIV's geneotype. The methods and devices disclosed herein significantly increase the availability of information to health care professionals and HIV infected persons for making informed choices regarding FTC drug therapy.

### 4.3 Identifying an NRTI-Resistant HIV-1

In certain aspects of the invention, methods are provided for determining whether an HIV-1 is likely to exhibit reduced susceptibility to NRTI therapy utilizing a genotype interpretation algorithm as described herein. In certain embodiments, the method comprises identifying the presence or absence of a primary mutation in a HIV-1 nucleic acid. In certain embodiments, the HIV-1 nucleic acid encodes HIV-1 reverse transcriptase.

In certain embodiments, the primary mutation is a mutation in the nucleic acid encoding codon 65, 151, or 184, or an insertion at codon 69 of HIV-1 reverse transcriptase. In certain embodiments, the primary mutation is a mutation at codon 65 of HIV-1 reverse transcriptase. In certain embodiments, the primary mutation is a mutation at codon 151 of HIV-1 reverse transcriptase. In certain embodiments, the primary mutation is a mutation at codon 184 of HIV-1 reverse transcriptase. In certain embodiments, the primary mutation is an insertion at codon 69 of HIV-1 reverse transcriptase. In certain embodiments, the primary mutation is K65R. In certain embodiments, the primary mutation is Q151M. In certain embodiments, the primary mutation is M184I. In certain embodiments, the primary mutation is M184V. In certain embodiments, the primary mutation is M184T. In certain embodiments, the primary mutation is any insertion of one or more amino acids at position 69. In certain embodiments, the primary mutation is selected from the group consisting of two, three, and four mutations selected from the group consisting of mutations in codons 65, 151, or 184, or an insertion at codon 69 of HIV-1 reverse transcriptase. In certain embodiments, the primary mutation is selected from the group consisting of two, three, four, five, and six mutations selected from the group consisting of K65R, Q151M, M184I, M184V, M184T, and any insertion of one or more amino acids at position 69.

In certain embodiments, the methods further comprise identifying the absence or presence of a secondary mutation in the HIV-1 nucleic acid. In certain embodiments, the secondary mutation is a mutation at codon 41, 44, 67, 70, 118, 210, 215, or 219 of HIV-1 reverse transcriptase. In certain embodiments, the secondary mutation is a mutation at codon 41 of HIV-1 reverse transcriptase. In certain embodiments, the secondary mutation is a mutation at codon 44 of HIV-1 reverse transcriptase. In certain embodiments, the secondary mutation is a mutation at codon 67 of HIV-1 reverse transcriptase. In certain embodiments, the secondary mutation is a mutation at codon 70 of HIV-1 reverse transcriptase. In certain embodiments, the secondary mutation is a mutation at codon 118 of HIV-1 reverse transcriptase. In certain embodiments, the secondary mutation is a mutation at codon 210 of HIV-1 reverse transcriptase. In certain embodiments, the secondary mutation is a mutation at codon 215 of HIV-1 reverse transcriptase. In certain embodiments, the secondary mutation is a mutation at codon 219 of HIV-1 reverse transcriptase. In certain embodiments, the secondary mutation is selected from the group consisting of any two, three, four, five, six, seven, and eight of codons selected from the group consisting of codon 41, 44, 67, 70, 118, 210, 215, and 219 of HIV-1 reverse transcriptase. In certain embodiments, the secondary mutation is M41L. In certain embodiments, the secondary mutation is E44A. In certain embodiments, the secondary mutation is E44D. In certain embodiments, the secondary mutation is D67N. In certain embodiments, the secondary mutation is K70R. In certain embodiments, the secondary mutation is V181. In certain embodiments, the secondary mutation is L210W. In certain embodiments, the secondary mutation is T215F. In certain embodiments, the secondary mutation is T215Y. In certain embodiments, the secondary mutation is K219E. In certain embodiments, the secondary mutation is K219H. In certain embodiments, the secondary mutation is K219N. In certain embodiments, the secondary mutation is K219Q. In certain embodiments, the secondary mutation is K219R. In certain embodiments, the secondary mutation is selected from the group consisting of any two, three, four, five, six, seven, eight, nine, ten, eleven, twelve, thirteen, and fourteen mutations selected from the group consisting of M41L, E44A, E44D, D67N, K70R, V118I, L210W, T215F, T215Y, K219E, K219H, K219N, K219Q and K219R.

In a preferred embodiment, the NRTI is FTC.

In certain embodiments, the HIV-1 is about 3.5 times less susceptible to FTC than that of a reference HIV-1. In certain embodiments, the reference HIV-1 is NL4-3. In certain embodiments, the HIV-1 is an HIV-1 isolated from a patient sample. In certain embodiments, the HIV-1 is isolated from the patient sample without passage through cell culture.

In certain embodiments, the HIV-1 determined to have a likelihood for reduced NRTI susceptibility exhibits a 3.5-fold change in a PHENOSENSE™ phentotypic HIV-1 assay compared to a reference HIV-1. In certain embodiments, the HIV-1 determined to have a likelihood for reduced NRTI susceptibility exhibits a 10-fold change in a PHENOSENSE™ phentotypic HIV-1 assay compared to a reference HIV-1.

In certain embodiments, the NRTI is FTC, the primary mutation is selected from the group consisting of K65R, Q151M, M184I, M184V, M184T, and any insertion of one or more amino acids at position 69, and the secondary mutations are selected from the group consisting of M41L, E44A, E44D, D67N, K70R, V118I, L210W, T215F, T215Y, K219E, K219H, L219N, K219Q and K219R.

In another aspect, the present invention provides a method for assessing the effectiveness of FTC therapy in a HIV-1-infected subject comprising detecting in a gene encoding reverse transcriptase of the HIV-1 one or more primary mutations or at least four secondary mutations, wherein the primary mutations are selected from the group consisting of mutations in codons 65, 151, and 184 and an insertion at codon 69, and the secondary mutations are selected from the group consisting of mutations at codons 41, 44, 67, 70, 118, 210, 215, and 219, thereby assessing the effectiveness of FTC therapy in the subject. In certain embodiments, the primary mutations are selected from the group consisting of K65R, Q151M, M184I, M184V, M184T, and any insertion of one or more amino acids at position 69. In certain embodiments, the secondary mutations are selected from the group consisting of M41L, E44A, E44D, D67N, K70R, V118I, L210W, T215F, T215Y, K219E, 219H, K219N, K219Q and K219R.

In certain embodiments, the decrease in susceptibility to FTC therapy is at least 3.5-fold. In certain embodiments, the decrease in susceptibility to FTC therapy is at least 10-fold.

The algorithms utilized in the methods of the invention have been developed by analysis and evaluation of the genotypes of a large dataset HIV-1 of known phenotypes to determine sets of reverse transcriptase mutations and combinations of these mutations that confer resistance to NRTIs. The following describes generally methods of generating genotype interpretation algorithms for the purpose of identifying drug resistant viruses.

4.3.1 Correlating Phenotypic and Genotypic Resistance to NRTIs

Datasets of viral variants with identified phenotypes can be used to correlate phenotypic and genotypic resistance to NRTIs.

Generally, a phenotypic analysis is performed and used to calculated the IC₅₀ or IC₅₀ of a drug for a virus variant. The results of the analysis can also be presented as fold-change in IC₅₀ or IC₉₀ for each variant as compared with a drug-susceptible reference virus or a viral sample taken from the same subject prior to a drug therapy.

Any method known in the art, without limitation, can be used to determine the phenotypic susceptibility or resistance of a mutant virus or population of viruses to an anti-viral therapy. Examples of determining phenotypes may found, for example, in U.S. Pat. Nos. 6,653,081, 6,489,098, 6,351,690, 6,242,187, 5,837,464, each of which is incorporated herein in its entirety for all purposes. For example, a phenotypic can be performed using the PHENOSENSE™ phenotype HIV assay (ViroLogic Inc., South San Francisco, Calif.). See Petropoulos et al., 2000, Antimicrob. Agents Chemother. 44:920-928, incorporated herein in its entirety for all purposes.

Any method known in the art can be used to determine whether a mutation is correlated with an increase in resistance of a virus to an NRTI. Typically, P values are used to determine the statistical significance of the correlation, such that the smaller the P value, the more significant the measurement. Preferably the P values will be less than 0.05 (or 5%). More preferably, P values will be less than 0.01. P values can be calculated by any means known to one of skill in the art. For the purposes of correlating an increase in resistance of an HIV-1 to a mutation, P values can be calculated using Fisher's Exact Test. See, e.g., David Freedman, Robert Pisani & Roger Purves, 1980, STATISTICS, W. W. Norton, New York. P values may be calculated using Student's paired and/or unpaired t-test and the non-parametric Kruskal-Wallis test (Statview 5.0 software, SAS, Cary, N.C.).

Resistance mutations in the HIV-1 reverse transcriptase gene are generally classified into two groups. A first group typically includes those mutations either selected first in the presence of the drug or are otherwise shown to have an effect on drug binding to the reverse transcriptase or an effect on viral activity and replication. A second group of mutations may include mutations that appear later than primary mutations and by themselves do not have a significant effect on resistance phenotype. This second group of mutations are frequently thought to improve replicative fitness caused by mutations of the first group.

Section 5.1 below provides additional details on the identification of an optimum set of HIV-1 reverse transcriptase mutations correlated to FTC resistance comprising a set of primary mutations (K65R, Q151M, M184I, M184V, M184T, and any insertion of one or more amino acids at position 69) and a set of secondary mutations (M41L, E44A, E44D, D67N, K70R, V118I, L210W, T215F, T215Y, K219E, K219H, K219N, K219Q and K219R).

In another aspect, the present invention provides a method for assessing the effectiveness of FTC therapy in a HIV-1-infected subject comprising detecting in a gene encoding reverse transcriptase of the HIV-1 one or more primary mutations or at least four secondary mutations, wherein the primary mutations are selected from the group consisting of mutations in codons 65, 151, and 184 and an insertion at codon 69, and the secondary mutations are selected from the group consisting of mutations at codons 41, 44, 67, 70, 118, 210, 215, and 219, thereby assessing the effectiveness of FTC therapy in the subject. In certain embodiments, the primary mutations are selected from the group consisting of K65R, Q151M, M184I, M184V, M184T, and any insertion of one or more amino acids at position 69. In certain embodiments, the secondary mutations are selected from the group consisting of M41L, E44A, E44D, D67N, K70R, V118I, L210W, T215F, T215Y, K219E, K219H, K219N, K219Q and K219R.

In certain embodiments, the decrease in susceptibility to FTC therapy is at least 3.5-fold.

Biological samples may include any sample that can contain an HIV, preferably an HIV-1. Biological samples from an HIV-infected subject include, for example and without limitation, blood, blood plasma, serum, urine, saliva, tissue swab and the like.

In certain embodiments, the one or more primary mutations encode an amino acid selected from the group consisting of K65R, Q151M, M184I, M184V, M184T, and any insertion of one or more amino acids at position 69. In certain embodiments, the one or more secondary mutations encode an amino acid selected from the group consisting of M41L, E44A, E44D, D67N, K70R, V118I, L210W, T215F, T215Y, K219E, K219H, K219N, K219Q and K219R.

Any method known to those of skill in the art may be used for detecting the presence or absence of a mutation in the reverse transcriptase of a HIV. The following section provides additional exemplary non-limiting guidance.

4.3.2 Detecting the Presence or Absence of Mutations in a Virus

The presence or absence of a viral mutation according to the present invention can be detected by any means known in the art for detecting a mutation. By “mutation” it is meant any variability in the nucleic acid sequence of a given HIV, or in the polypeptide sequence of the proteins of a given HIV, as compared to a reference HIV. Typically mutations of interest are those identified to confer resistance to a particular antiviral drug or combination of drugs, either existing alone or in a combination with other mutations. Thus, the mutation can be detected in the viral gene that encodes a particular protein, or in the protein itself, i.e., in the amino acid sequence of the protein.

In one embodiment, the mutation is in the viral nucleic acid. Such a mutation can be in, for example, a gene encoding a viral protein, in a cis or trans acting regulatory sequence of a gene encoding a viral protein, an intergenic sequence, or an intron sequence. The mutation can affect any aspect of the structure, function, replication or environment of the virus that changes its susceptibility to an anti-viral treatment. In one embodiment, the mutation is in a gene encoding a viral protein that is the target of an anti-viral treatment.

In another embodiment, the mutation is in a HIV-1 nucleic acid encoding a reverse transcriptase. For example, the mutation can be any mutation in codon 65, 151, 184, 41, 44, 67, 70, 118, 210, 215, or 219, or an insertion at codon 69. In one embodiment, the mutation in the nucleic acid encoding HIV-1 reverse transcriptase is selected from the group consisting of K65R, Q151M, M184I, M184V, M184T, M41L, E44A, E44D, D67N, K70R, V118I, L210W, T215F, T215Y, K219E, K219H, K219N, K219Q, K219R, and any insertion of one or more amino acids at position 69.

In certain embodiments, the mutation in the nucleic acid encoding HIV-1 reverse transcriptase is selected from the group consisting of K65R, Q151M, M184I, M184V, M184T, and any insertion of one or more amino acids at position 69.

In certain embodiments, the mutation in the nucleic acid encoding HIV-1 reverse transcriptase is selected from the group consisting of M41L, E44A, E44D, D67N, K70R, V118I, L210W, T215F, T215Y, K219E, K219H, K219N, K219Q, and K219R.

In certain embodiments, the mutation in the nucleic acid encoding confers a HIV phenotype resistant to FTC.

In certain embodiments, the mutation in a HIV nucleic acid encoding HIV-1 reverse transcriptase correlates with resistance to FTC as described herein.

A mutation within a viral gene can be detected by utilizing any method known by one of skill in the art without limitation. Viral DNA or RNA can be used as the starting point for such assay techniques, and may be isolated according to standard procedures which are well known to those of skill in the art.

The detection of a mutation in specific nucleic acid sequences, such as in a particular region of a viral gene, can be accomplished by a variety of methods including, but not limited to, restriction-fragment-length-polymorphism detection based on allele-specific restriction-endonuclease cleavage (Kan and Dozy, 1978, Lancet ii:910-912), mismatch-repair detection (Faham and Cox, 1995, Genome Res 5:474-482), binding of MutS protein (Wagner et al., 1995, Nucl Acids Res 23:3944-3948), denaturing-gradient gel electrophoresis (Fisher et al., 1983, Proc. Natl. Acad. Sci. U.S.A. 80:1579-83), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S.A. 92:87-91) cleavage of heteroduplex DNA, methods based on oligonucleotide-specific primer extension (Syvänen et al., 1990, Genomics 8:684-692), genetic bit analysis (Nikiforov et al., 1994, Nucl Acids Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids Res 23:675-682), radioactive or fluorescent DNA sequencing using standard procedures well known in the art, and peptide nucleic acid (PNA) assays (Orum et al., 1993, Nucl. Acids Res. 21:5332-5356; Thiede et al., 1996, Nucl. Acids Res. 24:983-984).

In addition, viral DNA or RNA may be used in hybridization or amplification assays to detect abnormalities involving gene structure, including point mutations, insertions, deletions and genomic rearrangements. Such assays may include, but are not limited to, Southern analyses (Southern, 1975, J. Mol. Biol. 98:503-517), single stranded conformational polymorphism analyses (SSCP) (Orita et al., 1989, Proc. Natl. Acad. Sci. USA 86:2766-2770), and PCR analyses (U.S. Pat. Nos. 4,683,202; 4,683,195; 4,800,159; and 4,965,188; PCR Strategies, 1995 Innis et al. (eds.), Academic Press, Inc.).

Such diagnostic methods for the detection of a gene-specific mutation can involve for example, contacting and incubating the viral nucleic acids with one or more labeled nucleic acid reagents including recombinant DNA molecules, cloned genes or degenerate samples thereof, under conditions favorable for the specific annealing of these reagents to their complementary sequences. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the virus can be immobilized, for example, to a solid support such as a membrane, or a plastic surface such as that on a microtiter plate or polystyrene beads. In this case, after incubation, non-annealed, labeled nucleic acid reagents of the type described above are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

Alternative diagnostic methods for the detection of gene specific nucleic acid molecules may involve their amplification, e.g., by PCR (U.S. Pat. Nos. 4,683,202; 4,683,195; 4,800,159; and 4,965,188; PCR Strategies, 1995 Innis et al. (eds.), Academic Press, Inc.), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Additionally, the nucleic acid can be sequenced by any sequencing method known in the art. For example, the viral DNA can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. See also the techniques described in Sambrook et al., 2001, Molecular Cloning: A Laboratory Manual, Cold Spring Harbor Laboratory, 3d ed., NY; and Ausubel et al., 1988 & updates, Current Protocols in Molecular Biology, John Wiley & Sons, NY.

The methods of the instant invention are applicable for determining resistance of an individual viral variant or for determining resistance of a variant population, which may be genotyped simultaneously. For example, for a given sequence, such as RT, sequencing a variant population together provides a genotype that can be used for identification of pertinent RT mutations.

Within the past decade, several technologies have been developed making it possible to identify large numbers (e.g., hundreds to hundreds of thousands) of nucleic acid sequences in a sample at any one time. See, e.g., Kozal et al., 1996, Nat. Med. 2(7):753-759; Lockhart et al., 1996, Nature Biotechnology 14:1675-1680; Blanchard et al., 1996, Nature Biotechnology 14, 1649; U.S. Pat. No. 5,571,639 issued Nov. 5, 1996. For example, a set oligonucleotide probes of predetermined sequences complimentary to various genotypes of a HIV reverse transcriptase can be attached to specific locations on a solid phase (an array), and the presence or absence of the various sequences in a unknown HIV nucleic acid sequence are determined by the hybridization patterns of the unknown HIV nucleic acid to the probes on the solid-phase. Typically, computer-aided techniques are used to assist in the gathering, processing, and evaluation of the large amount of information garnered in using array-based technology. See, e.g., U.S. Pat. No. 6,546,340 issued Apr. 8, 2003. Probe arrays including those made to custom specifications, along with reagents and computer analysis software are all commercially available (e.g., Affymetrix, Inc., Santa Clara Calif.).

Identification of a mutation in an HIV reverse transcriptase may be determined by amino acid analysis of the reverse transcriptase. Identification of a mutation in an HIV reverse transcriptase may be determined by the use of antibodies specifically recognizing particular amino residues at certain positions in HIV reverse transcriptase. Such antibodies can be used in ELISA assays or immunoprecipitation studies to assess the presence of mutant amino acids in the reverse transcriptase.

4.3.3 Applying an Classification Rule to a Genotype

The methods of the present invention apply certain selection rules upon the identified HIV-1 genotypes to classify a HIV-1 as being resistant (or less susceptible) to an NRTI or as being sensitive to an NRTI.

In one embodiment, the selection rule requires a condition to be met that one primary mutation or at least four secondary mutations are present in a nucleic acid encoding HIV-1 RT.

Any method known to those of skill in the art may employed to determined whether the condition as applied to a given HIV-1 is met. Typically, computers are employed that perform the function of determining whether the genotype of an HIV-1 meets the conditions for being classified as resistant to a drug. How a computer is programmed to determine whether a condition are met is not crucial to the practice of the instant invention as long as the condition for selecting resistant genotypes is properly applied. Thus, any type of computer and any type programming language known to those of skill in the are can be employed that can determine if a HIV-1 genotype meets a condition for being resistant to an NRTI.

In certain embodiments, the methods for assessing the effectiveness of FTC therapy in a HIV-1-infected subject comprise determining whether a nucleic acid of an HIV-1 infecting the subject contains a nucleic acid encoding HIV-1 reverse transcriptase having
(i) one or more primary mutations in the HIV-1 reverse transciptase at codon 65, 151, 184, or an insertion at codon 69, and
(ii) four or more secondary mutations in HIV-1 reverse transcriptase at codon 41, 44, 67, 70, 118, 210, 215, or 219
   wherein the presence of one primary mutation or at least four secondary mutations indicate that the HIV-1 is likely to be resistant to FTC, thereby assessing the effectiveness of FTC therapy.

Determining whether a nucleic acid contains one of the recited combination of mutations can be performed by any means known to those of skill in the art, without limitation. Any sequence of steps taken for making the determination, without limitation, may be taken so long as the recited combination can be determined. Thus, those of skill in the art recognize that no temporal order of steps for making the determination is intended by using the terms “primary mutation” and “secondary mutation” or by the order they are recited in an embodiment. For example, secondary mutations can be detected before primary mutations, or primary mutations can be detected before secondary mutations, or both primary and secondary mutations may be simultaneously detected.

As provided in Section 5, exemplary data indicates that the genotype interpretation algorithm can be applied in the methods of the invention for identifying an HIV-1 that is resistant to FTC. Because the genotyping interpretation rules were developed using a relevant clinical cutoff value, those of skill in the art recognize the immediate benefit that the methods of the instant invention can have in addressing whether a given HIV-1 will susceptible to FTC therapy in a subject infected with the HIV-1.

Thus, in certain embodiments, the identification of HIV-1 as being resistant to FTC indicates a decrease in susceptibility to FTC therapy about equal to or greater than a clinical cutoff value of 3.5.

4.3.4 Correlating Phenotypic and Genotypic Susceptibility

Any method known in the art can be used to determine whether a mutation is correlated with a decrease in susceptibility of a virus to an anti-viral treatment and thus is a resistance-associated mutation (“RAM”) according to the present invention. In one embodiment, P values are used to determine the statistical significance of the correlation, such that the smaller the P value, the more significant the measurement. Preferably the P values will be less than 0.05. More preferably, P values will be less than 0.01. P values can be calculated by any means known to one of skill in the art. In one embodiment, P values are calculated using Fisher's Exact Test. See, e.g., David Freedman, Robert Pisani & Roger Purves, 1980, STATISTICS, W. W. Norton, New York.

In a preferred embodiment, numbers of samples with the mutation being analyzed that have an IC₅₀ fold change below or above 2.5-fold are compared to numbers of samples without the mutation. A 2×2 table can be constructed and the P value can be calculated using Fisher's Exact Test. In such embodiments, P values smaller than 0.05 or 0.01 can be classified as statistically significant.

### 4.4 Constructing an Algorithm

In another aspect, the present invention provides a method of constructing an algorithm that correlates genotypic data about a virus with phenotypic data about the virus. In certain embodiments, the phenotypic data relate to the susceptibility of the virus to an anti-viral treatment. In certain embodiments, the anti-viral treatment is an anti-viral compound. In certain embodiments, the anti-viral compound is an NRTI. In certain embodiments, the NRTI is FTC.

In one embodiment, the method of constructing the algorithm comprises creating a rule or rules that correlate genotypic data about a set of viruses with phenotypic data about the set of viruses.

In one embodiment, a data set comprising genotypic and phenotypic data about each virus in a set of viruses is assembled. Any method known in the art can be used to collect genotypic data about a virus. Examples of methods of collecting such data are provided below. Any method known in the art can be used for collecting phenotypic data about a virus. Examples of such methods are provided below. In a preferred embodiment, the data set comprises one or more RAMs as described above. In certain embodiments, each genotypic datum is the sequence of all or part of a viral protein of a virus in the set of viruses. In certain embodiments, each genotypic datum in the data set is a single amino acid change in a protein encoded by the virus, relative to a reference protein in the reference virus. In certain embodiments, the genotype comprises two, three, four, five, six or more amino acid changes in the viral protein. In certain embodiments, the virus is HIV, and the protein is HIV reverse transcriptase. In a preferred embodiment, the virus is HIV-1. In certain embodiments, the reference protein is the reverse transcriptase from NL4-3 HIV.

In certain embodiments, each phenotypic datum in the data set is the susceptibility to an anti-viral treatment of a virus in the set of viruses. In certain embodiments, the anti-viral treatment is an anti-viral compound. In certain embodiments, the anti-viral compound is an NRTI. In a preferred embodiment, the NRTI is FTC. In certain embodiments, the susceptibility is measured as a change in the susceptibility of the virus relative to a reference virus. In certain embodiments, the susceptibility is measured as a change in the IC₅₀ of the virus relative to a reference virus. In certain embodiments, the change in IC₅₀ is represented as the fold-change in IC₅₀. In certain embodiments, the virus is HIV. In a preferred embodiment, the virus is HIV-1. In another preferred embodiment, the reference HIV is NL4-3 HIV.

The genotypic and phenotypic data in the data set can be represented or organized in any way known in the art. In certain embodiments, the data are displayed in the form of a graph. In certain embodiments of this type of representation, the y-axis represents the fold change in IC₅₀ of a virus in the data set relative to a reference virus. In certain embodiments, each point on the graph corresponds to one virus in the data set. In certain embodiments, the x-axis represents the number of mutations that a virus in the data set has. In certain embodiments, the position of the point indicates both the number of mutations and the fold change in anti-viral therapy treatment that the virus has, both measured relative to a reference strain. In certain embodiments, the genotypic and phenotypic data in the data set are displayed in the form of a chart.

In one aspect, an algorithm is formulated that correlates the genotypic data with the phenotypic data in the data set. In certain embodiments, a phenotypic cutoff point is defined. In a preferred embodiment, the phenotype is susceptibility to an anti-viral treatment. In certain embodiments, the phenotype is change in sensitivity to an anti-viral treatment relative to a reference virus, and the cutoff point is the value above which a virus or population of viruses is defined as phenotypically resistant (“PT-R”) to the anti-viral therapy and below which a virus or population of viruses is defined as phenotypically sensitive (“PT-S”) to the anti-viral therapy. In certain embodiments, the cutoff point is 2-fold, 2.5-fold, 3-fold, 5-fold, 10-fold, 15-fold, 20-fold, 30-fold, 40-fold, 50-fold or 100-fold greater than the IC₅₀ of a reference virus. In certain embodiments, the phenotypic cutoff point is the clinical cutoff value as defined above. In a preferred embodiment, the virus is HIV and the anti-viral therapy is treatment with an NRTI. In a preferred embodiment, the NRTI is FTC.

In certain embodiments, the phenotypic cutoff point is used to define a genotypic cutoff point. In certain embodiments, this is done by correlating the number of mutations in a virus of the data set with the phenotypic susceptibility of the virus. This can be done, for example, using a graph similar to one discussed above. A genotypic cutoff point can be selected such that most viruses having more than that number of mutations in the data set are phenotypically resistant (“PT-R”), and most viruses having fewer than that number of mutations are phenotypically sensitive (“PT-S”). By definition, a virus in the data set with number of mutations equal to, or more than the genotypic cutoff is genotypically resistant (“GT-R”) to the anti-viral treatment, and a virus in the data set with fewer than the genotypic cutoff number of mutations is genotypically sensitive (“GT-S”) to the anti-viral treatment. Thus, in certain embodiments, a genotypic cutoff point is selected that produces the greatest percentage of viruses in the data set that are either phenotypically resistant and genotypically resistant (“PT-R, GT-R”), or phenotypically sensitive and genotypically sensitive (“PT-S, GT-S”).

While this algorithm can provide a useful approximation of the relationship between the genotypic and phenotypic data in the data set, in most cases there will be a significant number of strains that are genotypically sensitive but phenotypically resistant (“GT-S, PT-R”), or genotypically resistant but phenotypically sensitive (“GT-R, PT-S”). Thus, in a preferred embodiment, the algorithm is further modified to reduce the percentage of discordant results in the data set. This can be done, for example, by removing from the data set each data point that corresponds to a virus population comprising a mixture of mutations including the wild-type, at a single position considered by the algorithm tested. This can have the effect of reducing the number of PT-S, GT-R results, thus lowering the overall percentage of discordant results and so improves the fit of the algorithm to a data set.

In certain embodiments, differential weight values are assigned to one or more mutations observed in the data set. An algorithm that does not include this step assumes that each mutation in the data set contributes equally to the overall resistance of a virus or population of viruses to an anti-viral therapy. For example, a mutation could be present in a data set that is almost always correlated with phenotypic resistance to an anti-viral treatment. That is, almost every virus that has the mutation is phenotypically resistant to the anti-viral treatment, even those strains having only one or two total mutations. In certain embodiments, such mutations are “weighted,” i.e., assigned an increased mutation score. A mutation can be assigned a weight of, for example, two, three, four, five, six, seven, eight or more. For example, a mutation assigned a weight of 2 will be counted as two mutations in a virus. Fractional weighting values can also be assigned. In certain embodiments, values of less than 1, and of less than zero, can be assigned, wherein a mutation is associated with an increased sensitivity of the virus to the anti-viral treatment.

One of skill in the art will appreciate that there is a tradeoff involved in assigning an increased weight to certain mutations. As the weight of the mutation is increased, the number of GT-R, PT-S discordant results may increase. Thus, assigning a weight to a mutation that is too great may increase the overall discordance of the algorithm. Accordingly, in certain embodiments, a weight is assigned to a mutation that balances the reduction in GT-S, PT-R discordant results with the increase in GT-R, PT-S discordant results.

In certain embodiments, the interaction of different mutations in the data set with each other is also factored into the algorithm. For example, it might be found that two or more mutations behave synergistically, i.e., that the coincidence of the mutations in a virus contributes more significantly to the resistance of the virus than would be predicted based on the effect of each mutation independent of the other. Alternatively, it might be found that the coincidence of two or more mutations in a virus contributes less significantly to the resistance of the virus than would be expected from the contributions made to resistance by each mutation when it occurs independently. Also, two or more mutations may be found to occur more frequently together than as independent mutations. Thus, in certain embodiments, mutations occurring together are weighted together. For example, only one of the mutations is assigned a weight of 1 or greater, and the other mutation or mutations are assigned a weight of zero, in order to avoid an increase in the number of GT-R, PT-S discordant results.

In another aspect, the phenotypic cutoff point can be used to define a genotypic cutoff point by correlating the number as well as the class of mutations in a virus of the data set with the phenotypic susceptibility of the virus. Examples of classes of mutations include, but are not limited to, primary amino acid mutations, secondary amino acid mutations, mutations in which the net charge on the polypeptide is conserved and mutations that do not alter the polarity, hydrophobicity or hydrophilicity of the amino acid at a particular position. Other classes of mutations that are within the scope of the invention would be evident to one of skill in the art, based on the teachings herein.

In certain embodiments, an algorithm is constructed that factors in the requirement for one or more classes of mutations. In certain embodiments, the algorithm factors in the requirement for a minimum number of one or more classes of mutations. In certain embodiments, the algorithm factors in the requirement for a minimum number of primary or secondary mutations. In certain embodiments, the requirement for a primary or a secondary mutation in combination with other mutations is also factored into the algorithm. For example, it might be found that a virus with a particular combination of mutations is resistant to an anti-viral treatment, whereas a virus with any mutation in that combination, alone or with other mutations that are not part of the combination, is not resistant to the anti-viral treatment.

By using, for example, the methods discussed above, the algorithm can be designed to achieve any desired result. In certain embodiments, the algorithm is designed to maximize the overall concordance (the sum of the percentages of the PT-R, GT-R and the PT-S, GT-S groups, or 100 minus (percentage of the PT-S, GT-R+PT-R, GT-S groups). In preferred embodiments, the overall concordance is greater than about 75%, 80%, 85%, 90% or 95%. In certain embodiments, the algorithm is designed to minimize the percentage of PT-R, GT-S results. In certain embodiments, the algorithm is designed to minimize the percentage of PT-S, GT-R results. In certain embodiments, the algorithm is designed to maximize the percentage of PT-S, GT-S results. In certain embodiments, the algorithm is designed to maximize the percentage of PT-R, GT-R results.

At any point during the construction of the algorithm, or after it is constructed, it can be further tested on a second data set. In certain embodiments, the second data set consists of viruses that are not included in the data set used to construct the algorithm, i.e., the second data set is a naive data set. In certain embodiments, the second data set contains one or more viruses that were in the data set used to construct the algorithm and one or more viruses that were not in that data set. Use of the algorithm on a second data set, particularly a naive data set, allows the predictive capability of the algorithm to be assessed. Thus, in certain embodiments, the accuracy of an algorithm is assessed using a second data set, and the rules of the algorithm are modified as described above to improve its accuracy. In a preferred embodiment, an iterative approach is used to create the algorithm, whereby an algorithm is tested and then modified repeatedly until a desired level of accuracy is achieved.

In one aspect, the construction or implementation of the algorithm can begin with a few “starting mutations” and proceed in steps in which it factors in the presence of certain mutations or classes of mutations. In one embodiment, the algorithm factors in the presence of one or more primary mutations, as described above, plus two secondary mutations. Any of the mutations listed above as secondary mutations can be used as secondary mutations. Next, the algorithm factors in other mutations in addition to the starting mutations. In certain embodiments, the algorithm, in all future stages, factors in a minimum number of secondary mutations. In a more particular embodiment, the algorithm, in all future stages, factors in at least 2 secondary mutations. When the algorithm factors in the combination of 2 or more mutations, it is generally understood that both mutations, e.g., 184V and 41L, be present in the same virus (or sample). Finally, the algorithm can factor in additional combinations, e.g., the combination of 184V or 41L with any one or more of, E44A, E44D, D67N, K70R, V118I, L210W, T215F, T215Y, K219E, or K219H. During the construction or implementation of an algorithm as described above, a decrease in the overall discordance as well as the percentage of data in the PT-R, GT-S group decreased with each step of the algorithm is indicative that the algorithm improved each time in correctly predicting the mutations and combinations of mutations that led to phenotypic resistance.

### 4.5 Using an Algorithm to Predict the Susceptibility of a Virus

In another aspect, the present invention also provides a method for using an algorithm of the invention to predict the phenotypic susceptibility of a virus or a derivative of a virus to an anti-viral treatment based on the genotype of the virus. In one embodiment, the method comprises detecting, in a viral nucleic acid or a nucleic acid prepared from a viral nucleic acid, the presence or absence of one or more RAMs, applying the rules of the algorithm to the detected RAMs, wherein a virus that satisfies the rules of the algorithm is genotypically resistant to the anti-viral treatment, and a virus that does not satisfy the rules of the algorithm is genotypically sensitive to the anti-viral treatment. In another embodiment, the method comprises detecting, in a viral nucleic acid or a nucleic acid prepared from a viral nucleic acid, the presence or absence of one or more RAMs, applying the rules of the algorithm to the detected RAMs, wherein a score equal to, or greater than the genotypic cutoff score indicates that the virus is genotypically resistant to the anti-viral treatment, and a score less than the genotypic cutoff score indicates that the virus is genotypically sensitive to the anti-viral treatment.

The algorithm of this invention can be used for any viral disease where anti-viral drug susceptibility is a concern, as discussed herein. In certain embodiments the assay of the invention can be used to determine the susceptibility of a retrovirus to an anti-viral drug. In a preferred embodiment, the retrovirus is HIV. Preferably, the virus is HIV-1.

The anti-viral agent of the invention could be any treatment effective against a virus. It is useful to the practice of this invention, for example, to understand the structure, life cycle and genetic elements of the viruses which can be tested in the drug susceptibility test of this invention. These would be known to one of ordinary skill in the art and provide, for example, key enzymes and other molecules at which the anti-viral agent can be targeted. Examples of anti-viral agents of the invention include, but are not limited to, nucleoside NRTIs such as AZT, ddI, ddC, d4T, 3TC, FTC, abacavir, nucleotide reverse transcriptase inhibitors such as tenofovir, NNRTIs such as nevirapine, efavirenz, delavirdine, fusion inhibitors such as T-20 and T-1249 and protease inhibitors such as saquinavir, ritonavir, indinavir, nelfinavir, amprenavir and lopinavir.

In some embodiments of the invention, the anti-viral agents are directed at retroviruses. In certain embodiments, the anti-viral agents are NRTIs such as AZT, ddI, ddC, d4T, 3TC, FTC, and abacavir. In certain embodiments, the anti-viral agents comprise two or more NRTIs. In certain embodiments, the NRTIs are administered in combination. In a preferred embodiment, the anti-viral agent is FTC.

Some mutations associated with reduced susceptibility to treatment with an anti-viral agent are known in the art. See, e.g., Maguire et al., 2002, Antimicrob Agents Chemother 46:731-738. Other such mutations are described herein.

### 4.6 Using an Algorithm to Predict the Effectiveness of Anti-Viral Treatment for an Individual

In another aspect, the present invention also provides a method for using an algorithm of the invention to predict the effectiveness of an anti-viral treatment for an individual infected with a virus based on the genotype of the virus. In certain embodiments, the method comprises detecting, in the virus or derivative of the virus, the presence or absence of one or more RAMs, applying the rules of the algorithm to the detected RAMs, wherein a virus that satisfies the rules of the algorithm is genotypically resistant to the anti-viral treatment, and a virus that does not satisfy the rules of the algorithm is genotypically sensitive to the anti-viral treatment, thereby identifying the effectiveness of the anti-viral treatment. In certain embodiments, the method comprises detecting, in the virus or a derivative of the virus, the presence or absence of one or more RAMs, applying the rules of the algorithm to the detected RAMs, wherein a score equal to, or greater than the genotypic cutoff score indicates that the virus is genotypically resistant to the anti-viral treatment, and a score less than the genotypic cutoff score indicates that the virus is genotypically sensitive to the anti-viral treatment.

As described in above, the algorithm of the invention can be used for any viral disease where anti-viral drug susceptibility is a concern and the anti-viral agent of the invention could be any treatment effective against a virus. In certain embodiments the assay of the invention is used to determine the susceptibility of a retrovirus to an anti-viral drug. In a preferred embodiment, the retrovirus is HIV. Preferably, the virus is HIV-1. In some embodiments of the invention, the anti-viral agents are directed at retroviruses In certain embodiments, the anti-viral agents are NRTIs such as AZT, ddI, ddC, d4T, 3TC, FTC, and abacavir. In certain embodiments, the anti-viral agents comprise two or more NRTIs. In certain embodiments, the NRTIs are administered in combination. In a preferred embodiment, the anti-viral agent is FTC.

As described above, mutations associated with reduced susceptibility to treatment with an anti-viral agent may be obtained from the art or determined by methods described herein.

In certain embodiments, the present invention provides a method for monitoring the effectiveness of an anti-viral treatment in an individual infected with a virus and undergoing or having undergone prior treatment with the same or different anti-viral treatment. In certain embodiments, the method comprises detecting, in a sample of the individual, the presence or absence of an amino acid residue associated with reduced susceptibility to treatment the anti-viral treatment, wherein the presence of the residue correlates with a reduced susceptibility to treatment with the anti-viral treatment.

### 4.7 Correlating Susceptibility to One Anti-Viral Treatment with Susceptibility to Another Anti-Viral Treatment

In another aspect, the present invention provides a method for using an algorithm of the invention to predict the effectiveness of an anti-viral treatment against a virus based on the genotypic susceptibility of the virus to a different anti-viral treatment. In certain embodiments, the method comprises detecting, in a virus or a derivative of a virus, the presence or absence of one or more mutations correlated with resistance to an anti-viral treatment and applying the rules of an algorithm of the invention to the detected mutations, wherein a virus that satisfies the rules of the algorithm is genotypically resistant to the anti-viral treatment, and a virus that does not satisfy the rules of the algorithm is genotypically sensitive to the anti-viral treatment. In certain embodiments, the method comprises detecting, in the virus or a derivative of the virus, the presence or absence of one or more mutations correlated with resistance to an anti-viral treatment and applying the rules of the algorithm to the detected mutations, wherein a score equal to, or greater than the genotypic cutoff score indicates that the virus is genotypically resistant to a different anti-viral treatment, and a score less than the genotypic cutoff score indicates that the virus is genotypically sensitive to a different anti-viral treatment. In certain embodiments, the anti-viral treatment is an NRTI. In certain embodiments, the NRTI is AZT, ddI, ddC, d4T, 3TC, FTC, or abacavir. In a preferred embodiment, the anti-viral agent is FTC. In certain embodiments, a mutation correlated with resistance to one NRTI is also correlated with resistance to another NRTI.

### 4.8 Computer Implemented Methods

In one aspect, the present invention provides a computer implemented method of identifying an HIV-1 as being less susceptible to FTC therapy in a subject infected with the HIV-1. Typically, data representing the HIV-1 genotype is received as input by a computer system. For example, data can be entered by a keyboard. As another example, data can be received electronically from a device used for the purpose of genotyping nucleic acid. Typically genotyping of HIV nucleic acid is resolved by electrophoretic methods using dye termination chemistry reactions, although other options are possible including hybridization patterns of a HIV nucleic acid to oligonucleotide array. Thus, the data received as input may represent electrophoretic migrations or hybridization patterns which can be converted into a representation of a genotype.

Embodiments of the computer implemented method comprise performing comparison of the genotype of the HIV-1 to a database representing pertinent NRTI resistance mutations. In preferred embodiments, the database comprises representations of mutant RT codons K65R, Q151M, M184I, M184V, M184T, M41L, E44A, E44D, D67N, K70R, V118I, L210W, T215F, T215Y, K219E, K219H, K19N, K219Q, K219R, and any insertion of one or more amino acids at position 69. Performing a comparison between the genotype of the HIV-1 and the database can be performed in any sequential order, without limitation, and does not depend on considerations such amino acid position in the reverse transcriptase or whether a particular position represents a site of a primary or secondary mutation; it is only required that performing a comparison is undertaken in such a way that the recited conditions can be determined.

In one aspect, the present invention provides a computer implemented method of determining that an HIV-1 is likely to be resistant to FTC, comprising inputting to a computer system a genotype of HIV-1 reverse transcriptase from the HIV-1 and comparing, thereby performing a first comparison, the genotype of the HIV-1 reverse transcriptase to a database in the computer system that comprises a correlation between the presence of a mutation at 65, 151, or 184 or an insertion at codon 69 and resistance to FTC, wherein if the comparison identifies that a mutation correlated with FTC resistance is present, the HIV-1 is determined to resistant to FTC. In certain embodiments, the mutation is K65R, Q151M, M184I, M184V, M184T, or any insertion of one or more amino acids at position 69.

In certain embodiments, the methods further comprise comparing, thereby performing a second comparison, the genotype of the HIV-1 reverse transcriptase to a database in the computer system that comprises a correlation between the presence of a mutation codon 41, 44, 67, 70, 118, 210, 215, or 219 and resistance to FTC, wherein if the comparison identifies that a mutation correlated with FTC resistance is present, the HIV-1 is determined to resistant to FTC. In certain embodiments, the mutation is M41L, E44A, E44D, D67N, K70R, V118I, L210W, T215F, T215Y, K219E, I<219H, K219N, K219Q or K219R.

In one embodiment, a computer implemented method comprises determining whether a condition is met that one match is made in the first comparison or at least four matches are made in the second comparison.

The computer implemented methods disclosed herein may implemented on any computer that is known to those of skill in the art, without limitation. It will be recognized that the implemented methods disclosed herein do not depend on a particular type of computer, memory storage elements, processing speeds, programming languages, compilers, other computer hardware, software or peripherals, and the like.

In certain embodiments, the computer implemented methods comprise displaying a result indicating whether or not that the HIV-1 is determined to be less susceptible to FTC therapy in a subject infected with the HIV-1. It is generally understood that an output device is used for the display of the results obtained using the computer-implemented methods of the invention. Output devices can be any type of printers, computer screens, disk drives, CD writers, other computers, or memory modules accessible by another computer, and the like, without limitation. Displaying a result can be any display known to those of skill in the art without limitation.

In certain embodiments, the result is displayed on a tangible medium. Typically, results are displayed on computer screens, printouts, CDs, and the like. In certain embodiments, the result is outputted as data on a tangible medium. In certain embodiments, the tangible medium is a paper, e.g., a computer printout. In certain embodiments, the tangible medium is a computer-readable memory, e.g., a random-access memory, a fixed disk drive, a floppy disk, a compact disk, an iPod™, a flash memory, etc.

### 4.9 Other Methods

Those of skill in the art recognize the value of providing the information that can be obtained using the methods disclosed herein. For example, costly yet ineffective antiviral drug treatment regimens can be avoided with the knowledge that an HIV-1 is resistant to an NRTI.

In one aspect, the present invention provides a method that comprises determining whether an HIV-1 is likely to be resistant to an NRTI according to a method of the invention, then providing information disclosing whether an HIV-1 taken from an HIV-1-infected subject is resistant to FTC. This information may provided to the subject or to a health care professional. In certain embodiments, the information further comprises informing the subject or health care professional of the treatment option of treating the subject with FTC. In certain embodiments, the information further comprises recommending that the subject or health care professional treat the subject with FTC. In certain embodiments, the information further comprises recommending that the subject or health care professional not treat the subject with FTC.

In one embodiment, the method comprises obtaining a genotype for nucleic acid encoding reverse transcriptase of the HIV-1. This can be performed, for example, by determining the genotype of the nucleic acid encoding reverse transcriptase from the HIV-1-infected subject and using techniques as described herein, or, for example by receiving genotypic information about the HIV-1's reverse transcriptase from another who genotyped the HIV-1.

In another embodiment, the method comprises identifying the presence or absence of a primary mutation in the HIV-1 reverse transcriptase that is K65R, Q151M, M184I, M184V, M184T, or any insertion of one or more amino acids at position 69 or identifying the presence or absence of at least four secondary mutations in HIV-1 reverse transcriptase selected from the group consisting of M41L, E44A, E44D, D67N, K70R, V118I, L210W, T215F, T215Y, K219E, K219H, K219N, K219Q and K219R. As previously explained, identifying the presence or absence of a primary or secondary mutation can be performed simultaneously or in any order.

In one embodiment, the method comprises determining whether a condition is met that the presence of one primary mutation or at least four secondary mutations are identified, such that if the condition is met, then the HIV-1 taken from the HIV-infected subject is resistant to FTC.

In one embodiment, the method further comprises preparing a tangible medium indicating whether the HIV-1 is resistant to FTC.

In one embodiment, the method further comprises conveying the tangible medium to the subject or a health care provider.

### 4.10 Devices and Systems

In another aspect, the present invention provides a computer system that is configured to perform the computer implemented methods described above. Computers are particular helpful in the performance of the instant methods given the amount of genotype data in combination with rapidity of computers in performing algorithms.

In one embodiment, the computer system comprises a desktop computer running Microsoft WINDOWS® operating system.

In another embodiment, the computer system comprises software written in PERL.

In another aspect, the present invention provides a paper display of the result produced by the methods disclosed herein.

In yet another aspect, the invention provides an article of manufacture that comprises computer-readable instructions for performing the computer-implemented methods discussed above. One embodiment is a CD. Another embodiment is an CD wherein the computer-readable instructions are in PERL.

In another aspect, the present invention provides a computer program product comprising one or more computer codes that identify an HIV-1 as being less susceptible to FTC treatment in a subject infected with HIV-1 and a computer readable medium that stores the computer codes. Several embodiments follow.

In one embodiment, the computer program comprises a computer code that receives input corresponding to the genotype of the HIV-1 nucleic acid encoding HIV-1 reverse transcriptase. The input may represent the nucleotide sequence of the HIV-1 nucleic acid, for example, a list of bases. The input may be converted from a hybridization pattern of the HIV-1 nucleic acid onto an oligonucleotide probe array attached to a solid phase. The input may be converted from an automated sequencer detecting electrophoretic migration.

In another embodiment, the computer program comprises a computer code that performs a first comparison to determine if an amino acid encoded by HIV-1 reverse transcriptase codons 65, 69, 151, and 184 matches one or more of mutant amino acids K65R, Q151M, M184I, M184V, M184T, and any insertion of one or more amino acids at position 69, and a computer code that performs a second comparison to determine if an amino acid encoded by HIV-1 reverse transcriptase codons 41, 44, 67, 70, 118, 210, 215, and 219 matches one or more of mutant amino acids M41L, E44A, E44D, D67N, K70R, V118I, L210W, T215F, T215Y, K219E, K219H, K219N, K219Q and K219R.

In another embodiment, the computer program comprises a computer code that determines whether a condition is met that one match is made in the first comparison or at least four matches are made in the second comparison, wherein the HIV-1 is identified as being less susceptible to FTC treatment if a condition is determined to be met.

In another embodiment, the computer program comprises a computer code conveys a result representing whether or not the HIV-1 is identified as being less susceptible to FTC treatment to an output device. An output device may any known to those of skill in the art, without limitation, such as a printer, a disk drive, a computer screen, another computer, and so forth.

In an aspect, the present invention provides a tangible medium storing the result conveyed to an output device as described above. A tangible medium may be any tangible medium known to those of skill in the art without limitation. A tangible medium may be a CD or DVD. A tangible medium may be a printout. A tangible may be a computer-readable medium as described above.

### 5. EXAMPLES

### 5.1 Example 1

### Defining a Set of Reverse Transcriptase Mutations and Numbers of Mutations to be Considered

A set of reverse transcriptase mutations for FTC was generated utilizing the HIPAA-compliant database of over 48,000 linked phenotype and genotype results for patient's samples maintained by ViroLogic, Inc. (South San Francisco, Calif.). Phenotypes and genotypes were determined in the ViroLogic clinical laboratory. Of these samples, 35,812 contained at least one drug-resistance associated mutation in reverse transcriptase or in protease (i.e., displayed evidence of exposure to one or more antiretroviral drugs). In this subset excluding the wild-type samples, a total of 13,576 samples had IC₅₀ fold change (FC) data available for FTC.

The drug susceptibility phenotypes of the HIV-1 isolates from patient plasma samples was determined by the PHENOSENSE™ phenotype HIV assay. This assay was performed by amplifying the PR-RT segment of the pol gene from patient plasma and inserting it into a genomic HIV-1 vector. The vector contained a luciferase reporter gene to monitor recombinant virus infection in cell culture. Results were expressed as the FC in the IC₅₀ for the patient-derived virus compared to that for a reference control virus, NL4-3. Drug dilutions were arranged to maximize curve-fitting accuracy for the range of wildtype virus susceptibilities over clinically relevant ranges of increased and decreased susceptibilities. Microtiter plates were incubated in customized incubators in which the temperature, CO₂ level, and humidity were controlled to minimize variation in cell growth and medium composition changes throughout the plate. Among the dataset of 13,576 samples with data available for FTC, 8,589 samples (63.2%) were classified as phenotypically resistant.

Genotypes were determined by the GENESEQ™ HIV assay. This assay uses the resistance test vectors constructed for the phenotype assay as the template, dye-terminator reaction chemistry, and automated capillary electrophoresis to determine the sequences of the patient-derived HIV-1 RTs (amino acids 1 to 305). The deduced amino acids sequences of patient viruses were compared to the sequence of NL4-3 (GenBank accession no. AF324493).

### 5.2 Example 2

### Discordance Rates for FTC Resistance Genotyping

In order to determine optimal rules for determining FTC resistance, a dataset (n=13,576) was culled from the database described above. This dataset was filtered to exclude wildtype (genotypes with no known mutations associated to resistance to NRTIs) and redundant samples.

Genotype interpretation algorithms were developed using PERL scripts, convenient for parsing text files. The programs were run on desktop computers running Microsoft WINDOWS® operating system.

Five rules were constructed to identify whether an HIV-1's genotype predicts resistance (GR) to FTC. The five rules include whether the HIV-1's reverse transcriptase contains:
The Q151M mutation (rule 1)
Or the T69* mutation (insertion of at least one amino acid at position 69) (rule 2)
Or the K65R mutation (rule 3)
Or the M184I, M184V or M184T mutations (rule 4)
Or 4 or more mutations among the following NAMs: M41L, E44A or D, D67N, K70R, V118I, L210W, T215F or Y, K219E, H, N, Q or R. (rule 5)

The five rules were tested on all 13,576 samples as a group and also separately by specific subgroup defined by mutations considered in each rule.

When all five rules were considered together, a sample was classified as GR to FTC when the conditions for any of the 5 rules was met. Otherwise the sample was classified as GS to FTC. The numbers of samples classified GR-PR, GS-PS, GR-PS, and GS-PR to FTC are shown in Table 1. The category “GR-PS-excluding mixtures” was defined for the samples with mutation not mixed at the position(s) of the rule being verified. This allows for a more accurate assessment of rule accuracy since the presence of mixtures is sometimes associated with a lower then expected FC value (see Parkin et al., 2002, J Acquir Immune Defic Syndr. 31(2):128-36).

TABLE 1

| Concordant | | | | | | | | |
|---|---|---|---|---|---|---|---|---|
| samples | | Discordant samples | | | | | | |
| | | | GRPS, no | | | | | |
| GSPS | GRPR | GRPS | mixtures | GSPR | N | GRPS % | GSPR % | disc % |
| 4,065 | 8,354 | 922 | 112 | 235 | 12,766 | 0.9% | 1.8% | 2.7% |

A total of 13,576 samples were analyzed. Among the 922 samples classified as GR-PS, 810 were found with a mixture at the key position of the rule being verified. They were excluded in the calculation of the proportions of discordant and concordant samples. Thus, the proportions were calculated using 12,766 samples as the denominator.

The five rules were tested separately and with the condition that none of the other rules were verified as true, except for rule 2. In other words, in this test, groups of samples were scored as genotypically resistant if they contained only one of the five mutation patterns and did not contain mutations scored in the other rules, except that samples with T69 insertions were allowed to have other NAMs. In this exercise, because of large number of samples in each category, mixtures (if present at accessory positions) were considered as mutant. In rule 2, because of the low number of samples with T69 insertion without NAMs, the condition was defined to select samples with T69 insertion and with no mutation Q151M, K65R, M184I, M184V or M184T. Overall results are shown in Table 2, below.

TABLE 2

| RULE | | PS | PR | Totals | PR % |
|---|---|---|---|---|---|
| 1 | Q151M | 6 | 7 | 13 | 53.8 |
| 2 | T69insertion (possible | 11 | 93 | 104 | 89.4 |
| | NAMs) | | | | |
| 3 | K65R | 7 | 140 | 147 | 95.2 |
| 4 | M184I, V, OR T | 0 | 1624 | 1624 | 100 |
| 5 | NAMs (rule 5) | 117 | 789 | 906 | 87.1 |
| | Totals | 142 | 2652 | 2794 | 94.9 |

In summary, application of the five rules to the analyzed samples which meet only one rule (except for insertions at codon 69 as discussed above) identified 94.9% of all samples determined to be PR. When all five rules were applied without regard to whether the sample satisfied more than one rule, the total discordance observed between the phenotypic and genotypic analysis was 2.7%.

The examples provided herein, both actual and prophetic, are merely embodiments of the present invention and are not intended to limit the invention in any way.

All publications and patent applications cited in this specification are herein incorporated by reference as if each individual publication or patent application were specifically and individually indicated to be incorporated by reference.

What is claimed is:

1. A method of determining whether an HIV-1 is likely to be resistant to a nucleoside reverse transcriptase inhibitor (“NRTI”), comprising detecting in a gene encoding reverse transcriptase of the HIV-1 one or more primary mutations or at least four secondary mutations, wherein the primary mutations are selected from the group consisting of mutations in codons 65, 151, and 184 and an insertion at codon 69, and the secondary mutations are selected from the group consisting of mutations at codons 41, 44, 67, 70, 118, 210, 215, and 219.

2. The method of claim 1, wherein the primary mutations are selected from the group consisting of K65R, Q151M, M184I, M184V, M184T, and any insertion of one or more amino acids at position 69.

3. The method of claim 1, wherein the secondary mutations are selected from the group consisting of M41L, E44A, E44D, D67N, K70R, V118I, L210W, T215F, T215Y, K19E, K219H, K219N, K219Q and K219R.

4. The method of claim 1, wherein the NRTI is FTC.

5. The method of claim 1, wherein the HIV-1 is an HIV-1 isolated from a patient sample.

6. The method of claim 5, wherein the HIV-1 is isolated from the patient sample without passage through cell culture.

7. The method of claim 1, wherein the NRTI is FTC, the primary mutation is selected from the group consisting of K65R, Q151M, M184I, M184V, M184T, and any insertion of one or more amino acids at position 69, and the secondary mutations are selected from the group consisting of M41L, E44A, E44D, D67N, K70R, V118I, L210W, T215F, T215Y, K219E, K219H, K219N, K219Q and K219R.

8. A method for assessing the effectiveness of FTC therapy in a HIV-infected subject comprising detecting in a gene encoding reverse transcriptase of the HIV-1 one or more primary mutations or at least four secondary mutations, wherein the primary mutations are selected from the group consisting of mutations in codons 65, 151, and 184 and an insertion at codon 69, and the secondary mutations are selected from the group consisting of mutations at codons 41, 44, 67, 70, 118, 210, 215, and 219, thereby assessing the effectiveness of FTC therapy in the subject.

9. The method of claim 8, wherein the primary mutations are selected from the group consisting of K65R, Q151M, M184I, M184V, M184T, and any insertion of one or more amino acids at position 69.

10. The method of claim 8, wherein the secondary mutations are selected from the group consisting of M41L, E44A, E44D, D67N, K70R, V118I, L210W, T215F, T215Y, K219E, K219H, K219N, K219Q and K219R.

11. A computer implemented method of determining that an HIV-1 is likely to be resistant to FTC, comprising inputting to a computer-readable medium a genotype of HIV-1 reverse transcriptase from the HIV-1 and comparing the genotype of the HIV-1 reverse transcriptase to a database that comprises a correlation between the presence of a mutation at 65, 151, or 184 or an insertion at codon 69 and resistance to FTC, wherein if the comparison identifies that a mutation correlated with FTC resistance is present, the HIV-1 is determined to resistant to FTC.

12. The method of claim 11, wherein the database comprises a correlation between the presence of K65R, Q151M, M184I, M184V, M184T, or any insertion of one or more amino acids at position 69 and FTC resistance.

13. The method of claim 11, further comprising comparing the genotype of the HIV-1 reverse transcriptase to a database in the computer system that comprises a correlation between the presence of a mutation in at least four of codons 41, 44, 67, 70, 118, 210, 215, and 219 and resistance to FTC, wherein if the comparison identifies that a mutation correlated with FTC resistance is present, the HIV-1 is determined to resistant to FTC.

14. The method of claim 13, wherein the database comprises a correlation between the presence of M41L, E44A, E44D, D67N, K70R, V118I, L210W, T215F, T215Y, K219E, K219H, K219N, K219Q or K219R and FTC resistance.

15. The method of claim 11 or 13, further comprising displaying whether or not the HIV-1 is determined to be resistant to FTC.

16. The method of claim 11 or 13, further comprising informing a medical professional whether the HIV-1 is resistant to FTC.

17. The method of claim 11 or 13, further comprising informing the subject whether the HIV-1 is resistant to FTC.

18. A computer-readable medium comprising a computer program that determines whether an HIV-1 infecting a subject is resistant to FTC, comprising a computer code that receives input corresponding to a genotype of a HIV-1 nucleic acid encoding HIV-1 reverse transcriptase obtained from HIV-1 infecting the subject; a computer code that performs a first comparison to determine if codon 65, 69, 151, or 184 of the nucleic acid encoding HUV-1 reverse transcriptase is K65R, Q151M, M184I, M184V, M184T, or any insertion of one or more amino acids at position 69; a computer code that performs a second comparison to determine if codon 41, 44, 67, 70, 118, 210, 215, or 219 of the nucleic acid encoding HIV-1 reverse transcriptase is M41L, E44A, E44D, D67N, K70R, V118I, L210W, T215F, T215Y, K219E, K219H, K219N, K219Q or K219R; a computer code that determines whether at least one match is made in the first comparison or at least four matches are made in the second comparison, wherein the HIV-1 is determined to be resistant to FTC if at least one match is made in the first comparison or at least four matches are made in the second comparison; and a computer code that conveys a result representing whether or not the HIV-1 is determined to be resistant to FTC to an output device.
